(19) United States
(12) Patent Application Publication
(10) Pub. No.: US 2010/0034861 A1
(43) Pub. Date: Feb. 11, 2010

(54) DISPERSIONS OF NANOUREAS COMPRISING BIOLOGICALLY ACTIVE COMPOUNDS

(75) Inventors: Sebastian Dörr, Duesseldorf (DE); Michael Mager, Leverkusen (DE); Burkhard Köhler, Zierenberg (DE); Heike Heckroth, Odenthal (DE)

Correspondence Address: CONNOLLY BOVE LODGE & HUTZ, LLP, P O BOX 2207, WILMINGTON, DE, 19899, US

(73) Assignee: Bayer MaterialScience AG, Leverkusen (DE)

(21) Appl. No.: 12/579,003

(22) Filed: Oct. 14, 2009

Related U.S. Application Data
(62) Division of application No. 11/891,973, filed on Aug. 14, 2007.

(30) Foreign Application Priority Data
Aug. 18, 2006 (DE) 10 2006 038 940.9

(51) Int. Cl. A61K 9/14; B05D 3/00; A61K 47/16; A61K 9/00; A61P 43/00

(52) U.S. Cl. 424/422; 427/2.1; 514/788; 424/489

(57) ABSTRACT

The present invention relates to dispersions of nanoureas comprising biologically active compounds, a process for their preparation, and their use.

### CROSS REFERENCE TO RELATED APPLICATION

This application claims priority under 35 U. S.C. § 119 (a-d) to German application 10 2006 038 940.9, filed Aug. 18, 2006.

### FIELD OF THE INVENTION

The present invention relates to dispersions of nanoureas comprising biologically active compounds, a process for their preparation, and their use.

### BACKGROUND OF THE INVENTION

The finishing of a plastic with a biologically active compound often fails because of the fact that the active compound is incompatible with the plastic and therefore homogeneous incorporation is not possible. The locally differing active compound concentration is a great disadvantage, as by means of this areas result which are free of active compound and thus inactive. Moreover, the mechanical properties of the plastic can be very disadvantageously influenced in the case of inhomogeneous incorporation.

If the plastic is to be modified by dissolving the active compound and mixing it with the (optionally likewise dissolved) plastic, this process has the following disadvantages: on the one hand, equally suitable solvents cannot be found for all active compounds and plastics; on the other hand, the use of organic solvents is in principle disadvantageous, inter alia on account of the fact that residues thereof can remain in the product, which would not be acceptable, for example, for medicotechnological articles.

The finishing of plastics with biologically active compounds, however, is important, in particular in the field of medical devices. For instance, the bacterial colonization of medical devices such as catheters is a great problem, because this is often the initial step for subsequent, serious infection of the patient treated. Numerous processes for the antimicrobial finishing of catheters have therefore been proposed, both the finishing of the catheter material itself (e.g. of silicone, polyurethane, latex or PVC), and coating with an antimicrobially active material being possible. As antimicrobially active coatings, it has been proposed, for example, to deposit a pure metal layer of doped silver (U.S. Pat. No. 5,320,908, U.S. Pat. No. 5,395,651 & U.S. Pat. No. 5,965,204); the adhesion of these (friable) coatings to the catheter material, however, is poor. Coatings of special inorganic glasses which release Ag, Cu or Zn ions by hydrolysis of the glass are a further proposal (U.S. Pat. No. 6,143,318). Mixtures of Ag salts with sulphonamides (U.S. Pat. No. 4,581,028) or triclosan (WO 2000/57933), and the use of metal colloids have furthermore been proposed. In addition to the disadvantages already described, all aforementioned coatings, however, moreover have the disadvantage that the release of the active compound, in the examples cited Ag ions, is not constant in terms of time, i.e. the Ag ions are eluted very rapidly at the beginning, then the release decreases considerably and the antimicrobial activity is lost. Compensation of the effect by appropriate increase in the initial active compound concentration is not possible, as undesired side effects can thereby occur. In the case of catheters, for example, frequent exchange is therefore necessary in order to reduce the microbial contamination.

DE-A 697 34 168 describes implants having a hollow space which contains active compounds and slowly releases these. This form of encapsulation is very involved and the implant must be inserted by means of a surgical intervention. Transfer of this method of resolution to coatings or plastics is not possible with a macroscopic “slow release” system of this type.

In DE-A 10 2004 030504, the use of pH-sensitive polymers for the coating of macroscopic, oral pharmaceutical forms for selective active compound release is described. The applicability is restricted to areas in which release of the active compounds is to be brought about by selective change of the pH in the environment.

In DE-A 698 19 145, biodegradable polymers are employed in order to encase active compounds. The problem is the lack of stability of compounds of this type in aqueous systems on account of their susceptibility to hydrolysis or microbial degradation.

DE 4122591 describes microparticles of water-insoluble polymers, which are dispersed in water and consolidated using a gelling agent. On subsequent drying, polymer pellets are obtained. The disadvantages are a very involved preparation process and the incompatibility of the microparticles with many additives from galenics such as surfactants or ionically charged polymers.

DE 19930795 describes the encapsulation of active compounds by diffusion into polymer beads of 50 to 2000 μm diameter. The use of polylactates in turn leads to a system which is not stable on storage in the presence of moisture and to instability to microorganisms.

In EP 0429187, slow-release formulations of crosslinked polyvinylpyrrolidones are described which contain a certain type of steroids and can release with a delay. The process described is restricted to the use of a certain class of steroids.

All systems described are suitable only for specific application systems and certain classes of active compound. A process with which a broad spectrum of active compounds can be covered is not described. Furthermore, the production of the respective systems is in general involved and in some cases does not make possible the complete separation of solvents used. Incorporation into plastics or coatings is not described.

The preparation of aqueous nanourea dispersions containing urea particles of a size from 10 to 400 nm is known in principle and described, for example, in WO 2005/063873. In this process, hydrophilized polyisocyanates are added to water, optionally in the presence of a catalyst, whereby crosslinkage within the essentially dispersed particles takes place by means of urea bonds. To what extent such dispersions are compatible with active compounds and/or can be employed for the modification of plastics which show controlled release behaviour of the active compounds contained therein is not described.

### SUMMARY OF THE INVENTION

The object of the present invention was therefore the making available of an active compound-compatible plastic matrix, from which both coatings and materials and moulded articles can be produced and which shows “controlled release behaviour”, that is controlled release characteristics, optionally delayed over a period of time.

It has now been found that this object can be achieved by special nanourea dispersions which contain active compounds intended for release.

The present invention therefore relates to a process for the preparation of active compound-containing, aqueous nanourea dispersions, in which
A) nanoureas are formed by reaction of hydrophilized polyisocyanates in an aqueous medium with the formation of urea structures —NH—C(O)—NH—, where
B) at least one active compound is added before, during or after the urea formation in A.

Below, biologically active compounds are defined as elements or chemical compounds which have an action on living systems, in particular prions, viruses, bacteria, cells, fungi and organisms.

### DETAILED DESCRIPTION OF THE INVENTION

As used herein in the specification and claims, including as used in the examples and unless otherwise expressly specified, all numbers may be read as if prefaced by the word “about”, even if the term does not expressly appear. Also, any numerical range recited herein is intended to include all sub-ranges subsumed therein.

Examples of biologically active compounds include are biocidal compounds which have, for example, pesticidal, fungicidal, algicidal, insecticidal, herbicidal, spermicidal, parasiticidal, antibacterial (bacteria-destroying), bacteriostatic, antibiotic, antimycotic (fungi-destroying); antiviral (virus-destroying), virostatic and/or antimicrobial (microbe-destroying) action. Active compound combinations and combination, for example, with excipients, binders, neutralizing agents or additives are also possible. Other active compounds and combinations, for example active compounds from the field of human medicine or veterinary medicine, can also be employed.

As hydrophilized polyisocyanates, all NCO group-containing compounds known to the person skilled in the art can be employed per se which are non-ionically, (potentially) anionically or (potentially) cationically hydrophilized. Preferably, the hydrophilized polyisocyanates have at least one non-ionically hydrophilized structural unit. Particularly preferably, the hydrophilization of the polyisocyanates is carried out exclusively by non-ionically hydrophilizing groups.

Such non-ionically hydrophilizing groups are preferably introduced into polyisocyanates by reaction with polyethers, these polyethers preferably being monofunctional with respect to groups contained therein which are reactive to NCO groups. Examples of such NCO-reactive groups are hydroxyl, thiol or amino functions. In principle, however, these can also contain more than one NCO-reactive group.

The polyethers of the aforementioned type employed for hydrophilization are typically polyoxyalkylene ethers, in which preferably 30% by weight to 100% by weight of the oxyalkylene units are oxyethylene groups and up to 70% by weight are oxypropylene units.

Particularly preferably, they correspond to the type mentioned above and have, on statistical average, 5 to 70, preferably 7 to 55, oxyethylene groups per molecule.

Such polyethers are accessible in a manner known per se by alkoxylation of suitable starter molecules (e.g. in Ullmanns Encyclopädie der technischen Chemie [Ullmann's Encyclopaedia of Industrial Chemistry], 4th Edition, Volume 19, Verlag Chemie, Weinheim pp. 31-38).

Suitable starter molecules are, for example, saturated monoalcohols such as methanol, ethanol, n-propanol, isopropanol, n-butanol, isobutanol, see-butanol, the isomeric pentanols, hexanols, octanols and nonanols, n-decanol, n-dodecanol, n-tetradecanol, n-hexadecanol, n-octadecanol, cyclohexanol, the isomeric methyl-cyclohexanols or hydroxymethylcyclohexane, 3-ethyl-3-hydroxymethyloxetane or tetrahydrofurfuryl alcohol, diethylene glycol monoalkyl ethers such as, for example, diethylene glycol monobutyl ether, unsaturated alcohols such as allyl alcohol, 1,1-dimethylallyl alcohol or oleyl alcohol, aromatic alcohols such as phenol, the isomeric cresols or methoxyphenols, araliphatic alcohols such as benzyl alcohol, anisyl alcohol or cinnamyl alcohol, secondary monoamines such as dimethylamine, diethylamine, dipropylamine, diisopropylamine, dibutylamine, bis(2-ethylhexyl)amine, N-methyl- and N-ethylcyclohexylamine or dicyclohexylamine and heterocyclic secondary amines such as morpholine, pyrrolidine, piperidine or 1H-pyrazole. Preferred starter molecules are saturated monoalcohols. Particularly preferably, methanol, butanol and diethylene glycol monobutyl ether are used as the starter molecule.

Alkylene oxides suitable for the alkoxylation reaction are in particular ethylene oxide and propylene oxide, which can be employed in the alkoxylation reaction in any desired sequence or alternatively as a mixture.

The hydrophilized polyisocyanates are based on aliphatic, cycloaliphatic, araliphatic and aromatic polyisocyanates known per se to the person skilled in the art and having more than one NCO group per molecule and an isocyanate content of 0.5 to 50, preferably 3 to 30, particularly preferably 5 to 25,% by weight or their mixtures.

Examples of such suitable polyisocyanates are butylene diisocyanate, tetramethylene diisocyanate, cyclohexane-1,3- and 1,4-diisocyanate, hexamethylene diisocyanate (HDI), 1-isocyanato-3,3,5-trimethyl-5-isocyanatomethylcyclohexane (isophorone diisocyanate, IPDI), 2,4,4-trimethylhexamethylene diisocyanate, isocyanatomethyl-1,8-octanediisocyanate, methylenebis(4-isocyanatocyclohexane), tetramethylxylylene diisocyanate (TMXDI) or triisocyanatononane (TIN, 4-isocyanatomethyl-1,8-octane diisocyanate) and optionally also mixtures with other di- or polyisocyanates. In principle, aromatic polyisocyanates such as 1,4-phenylene diisocyanate, 2,4- and/or 2,6-toluylene diisocyanate (TDI), diphenylmethane-2,4′- and/or 4,4′-diisocyanate (MDI), triphenylmethane-4,4′-diisocyanate, naphthylene-1,5-diisocyanate are also suitable.

In addition to the aforementioned polyisocyanates, higher molecular weight secondary products with uretdione, isocyanurate, urethane, allophanate, biuret, iminooxadiazinedione and/or oxadiazinetrione structure can also be employed. Such secondary products are known in a known manner from the monomeric diisocyanates modification reactions which are known and described, for example, in Laas et al., J. prakt. Chem., 336, 1994, 185-200.

Preferably, the hydrophilized polyisocyanates of component A) are based on polyisocyanates or polyisocyanate mixtures of the aforementioned type having exclusively aliphatically or cycloaliphatically bonded isocyanate groups or their arbitrary mixtures.

Particularly preferably, the hydrophilized polyisocyanates are based on hexamethylene diisocyanate, isophorone diisocyanate or the isomeric bis(4,4′-isocyanatocyclohexyl)methanes and mixtures of the aforementioned diisocyanates.

Catalysts can additionally be used for the preparation of the nanourea dispersions. Those suitable are, for example, tertiary amines, tin, zinc or bismuth compounds or basic salts.

Suitable tertiary amines are triethylamine, tributylamine, dimethylbenzylamine, dicyclohexylmethylamine, dimethylcyclohexylamine, N,N,N′,N′-tetramethyldiaminodiethyl ether, bis-(dimethylaminopropyl)urea, N-methyl- and N-ethylmorpholine, N,N′-dimorpholinodiethyl ether (DMDEE), N-cyclohexylmorpholine, N,N,N′,N′-tetramethylethylenediamine, N,N,N′,N′-tetramethylbutanediamine, N,N,N′,N′-tetramethylhexane-1,6-diamine, pentamethyldiethylenetriamine, dimethylpiperazine, N-dimethylaminoethylpiperidine, 1,2-dimethylimidazole, N-hydroxypropylimidazole, 1-azabicyclo-(2,2,0)-octane, 1,4-diazabicyclo-(2,2,2)-octane (Dabco) and alkanolamine compounds, such as triethanolamine, triisopropanolamine, N-methyl- and N-ethyldiethanolamine, dimethylaminoethanol, 2-(N,N-dimethylaminoethoxy)ethanol, N,N′,N-tris-(dialkylaminoalkyl)hexahydrotriazines, e.g. N,N′,N-tris(dimethylaminopropyl)-s-hexahydrotriazines, iron(II) chloride, zinc chloride or lead octoate.

Tertiary amines of the aforementioned type, tin salts, such as tin dioctoate, tin diethylhexoate, dibutyltin dilaurate and/or dibutyldilauryltin mercaptide 2,3-dimethyl-3,4,5,6-tetrahydropyrimidine, tetraalkylammonium hydroxides, such as tetramethylammonium hydroxide, alkali metal hydroxides, such as sodium hydroxide, alkali metal alkoxides, such as sodium methoxide and potassium isopropoxide and/or alkali metal salts of long-chain fatty acids having 10 to 20 carbon atoms and optionally lateral OH groups are preferred.

Particularly preferred catalysts are tertiary amines; triethylamine, ethyldiisopropylamine and 1,4-diazabicyclo[2,2,2] octane are very particularly preferred.

These catalysts are typically employed in amounts of 0.01 to 8% by weight, preferably of 0.05 to 5% by weight, particularly preferably of 0.1 to 3% by weight, based on the total solids content of the resulting dispersion. Mixtures of the catalysts can also be added.

It is possible to add to the mixture solvents such as, for example, N-methylpyrrolidone, N-ethylpyrrolidone, methoxypropyl acetate, dimethyl sulphoxide, methyloxypropyl acetate, acetone and/or methyl ethyl ketone. After termination of the reaction and dispersion, volatile solvents such as acetone and/or methyl ethyl ketone can be removed by distillation. Preparation without solvents and the use of acetone or methyl ethyl ketone is preferred; preparation without solvents is particularly preferred.

For the preparation of the dispersions, the hydrophilized polyisocyanates described above are dispersed in an aqueous medium, optionally in the presence of catalysts.

Dispersion and reaction are preferably carried out by means of thorough mixing by a stirrer or other types of thorough mixing such as recirculation, static mixer, spike mixer, nozzle jet disperser, rotor and stator or under the influence of ultrasound.

In principle, during or after the dispersion, a modification of NCO groups with isocyanate-reactive compounds such as primary or secondary amines and/or (poly) alcohols can be carried out.

Preferably, the molecular ratio of NCO groups of the hydrophilized polyisocyanate to water is 1:100 to 1:5, particularly preferably 1:30 to 1:10.

In principle, it is possible to disperse the hydrophilized polyisocyanate in the water in one portion. Continuous addition of the hydrophilized polyisocyanate, for example over a period of 30 minutes to 20 hours, is also possible. However, addition in portions is preferred, the number of portions being 2 to 50, preferably 3 to 20, particularly preferably 4 to 10, where the portions can be of identical or alternatively different size.

The waiting time between the individual portions is typically 5 minutes to 12 hours, preferably 10 minutes to 8 hours, particularly preferably 30 minutes to five hours.

Continuous addition of the hydrophilized polyisocyanate over a period of 1 hour to 24 hours, preferably 2 hours to 15 hours is likewise preferred.

In the urea particle preparation, the vessel temperature is typically 10 to 80° C., preferably 20 to 70° C. and particularly preferably 25 to 50° C.

Preferably, following the reaction of hydrophilized polyisocyanate and water, the reactor is evacuated at internal temperatures of 0° C. to 80° C., preferably 20° C. to 60° C. and particularly preferably 25° C. to 50° C. The evacuation is carried out up to an internal pressure of 1 to 900 mbar, preferably 10 to 800 mbar, particularly preferably 100 to 400 mbar. The duration of this degassing subsequent to the actual reaction is typically 1 minute to 24 hours, preferably 10 minutes to 8 hours. Degassing is also possible by means of temperature increase without evacuation.

Preferably, simultaneously to the evacuation the nanourea dispersion is thoroughly mixed, e.g. by stirring.

The solids content of the urea particles present in the dispersion obtained according to A) is typically 10 to 60% by weight preferably 20 to 50% by weight, particularly preferably 30 to 45%.

The incorporation of the active compounds can be carried out during or after the particle preparation. For this, the active compounds can be present even during the dispersion of the hydrophilized polyisocyanate or can be metered parallel to this or added after the preparation of the particles. At least partial absorption of the active compound in the particles occurs here. This absorption in the interior and/or on the surface of the particles leads to time-distributed release characteristics of the active compound.

If the active compound added is not completely dissolved or absorbed into the dispersion, residual active compound can be separated off, for example by filtration.

In order to remove active compounds dissolved in the dispersion water, not bonded to the nanourea, the dispersion can be freed of low molecular weight constituents, for example, by dialysis or ultrafiltration according to processes known per se. The particular exclusion limit of the membrane is to be chosen here according to the hydrodynamic volume of the dissolved active compound. Preferred exclusion limits are less than 1 000 000 Daltons (=g/mol), particularly preferably less than 100 000 Daltons (=g/mol).

Typically, the amount of active compound, based on the solids content of the urea particles present, is 0.0001 to 50% by weight, preferably 1 to 20% by weight, particularly preferably 5 to 15% by weight. The amount of the active compounds in general depends on the amount of the particular active compound needed for the particular indication.

Active compounds which are only poorly or not soluble whatsoever in water are preferably mixed with the hydrophilized polyisocyanate optionally with the aid of co-solvents and subsequently dispersed in the aqueous medium. Preferably, these active compounds, however, contain no NCO-reactive groups or if they do contain such groups, the reaction to give the urea must be designed such that a noticeable reaction of the NCO groups with the active compound does not occur. If solvents are employed for the incorporation of active compounds, they are preferably removed again by distillation following the incorporation.

In the incorporation of the active compounds, temperatures of 25 to 100° C. are preferably chosen.

In the process according to the invention, it is additionally possible, of course, to use excipients and additives such as, for example stabilizers, surfactants, solubilizers, neutralizing agents, trapping reagents for reactive groups, flow auxiliaries and/or free-radical scavengers.

The dispersions obtainable by the process according to the invention and the active compound-containing nanoscale urea particles contained therein are a further subject of the invention.

These nanourea particles have an average particle size determined by means of laser correlation spectroscopy of 10 to 300 nm, preferably 20 to 150 nm.

These active compound-containing nanourea dispersions can also be dried by methods customary per se in the industry, such as distillation, freeze drying or spray drying.

Both the dispersions obtained according to the invention and the particles contained therein are valuable starting materials for the production of active compound-containing coatings, materials and moulded articles which are preferably based on polyurethanes.

For incorporation in a coating formulation, the active compound-containing nanourea dispersions can be employed as such, in particular if the coating formulation itself contains constituents dispersed in water, such as, for example, the binder.

However, it is also possible to dry the dispersions and to incorporate the active compound-containing nanoureas as a solid. Incorporation of the nanoureas with active compounds in a solvent is also possible.

Preferred binders in such coating formulations are polyurethanes, poly(meth)acrylates, polyesters and silicones of all types. Polyurethanes which can be employed in the form of aqueous dispersions, solutions in organic solvents or also free of solvents are particularly preferred. Single- and two-component polyurethanes can similarly be employed here.

This coating formulation can be applied to an article in any desired manner, for example by spraying, vapourizing, brushing, immersing, flooding or with the aid of rollers and doctor blades. Suitable substrates are, for example, metals, plastics, in particular polyethylene, polypropylene, polytetrafluoroethylene, polyurethanes, silicones, polyvinyl chloride, poly(meth)acrylates, polycarbonates, polyesters, wood, material, fabric or glass. The application of the coating formulation and the drying and/or hardening can be carried out before, during or after shaping of the article. The drying and/or hardening is carried out at room temperature or elevated temperatures, optionally under reduced pressure.

Materials and moulded articles which can be produced with the aid of the particles and dispersions according to the invention or can be coated with coatings comprising the particles according to the invention are all commodities known per se, in which microbial exposure occurs, for example, due to frequent contact (e.g. handles of any type), but articles for storage, transport (e.g. pipes) or processing of liquid media can also be understood among these. However, articles from the medicotechnology field such as, for example, catheters, tubes, vessels, orifices, implants, artificial organs (outside and inside the body), protheses, vascular protheses (stents), visual aids (e.g. contact lenses), endoscopes and wound coverings are preferred.

### EXAMPLES

If not noted differently, all percentages refer to percentage by weight.

If not noted differently, all analytical measurements refer to temperatures of 23° C.

The stated viscosities were determined by means of rotary viscometry according to DIN 53019 at 23° C. using a rotary viscometer from Anton Paar Germany GmbH, Ostfildern, DE.

If not expressly mentioned otherwise, NCO contents were determined volumetrically according to DIN-EN ISO 11909.

The stated particle sizes were determined by means of laser correlation spectroscopy (apparatus: Malvern Zetasizer 1000, Malver Inst. Limited).

The solids contents were determined according to DIN-EN ISO 3251.

Checking for free NCO groups was carried out by means of IR spectroscopy (band at 2260 cm⁻¹).

The concentrations of the silver ions were determined spectroscopically according to DIN ISO 17025.

Dialyses were carried out using the Float-A-Lyzer® floating dialysis tubes of Spectra/Por®. The tube material was cellulose ester membranes having a nominal exclusion limit of 25 000 g/mol. The membranes were rinsed with deionized water before use and conditioned in a water bath.

### Chemicals

Bayhydur® VP LS 2336: hydrophilized polyisocyanate based on hexamethylene diisocyanate, solvent-free, viscosity about 6800 mPas, isocyanate content about 16.2%, Bayer MaterialScience AG, Leverkusen, DE.
Impranil® DLN Anionically hydrophilized, uncrosslinked, aliphatic polyesterpolyurethane dispersion in water having a solids content of about 40%) Bayer MaterialScience AG, Leverkusen, DE.
Isofoam® 16: antifoam agent, Petrofer-Chemie, Hildesheim, DE.
The other chemicals were obtained from the fine chemicals business of Sigma-Aldrich GmbH, Taufkirchen, DE.

### Active Compounds:

| | | Active | |
|---|---|---|---|
| | | compound | |
| | Active compound | content | Source |
| 1 | Ciprofloxacin | 100% | Bayer HealthCare AG, |
| | | | Leverkusen, DE |
| 2 | Acetylsalicylic acid | 100% | Sigma-Aldrich Chemie GmbH, |
| | | | Taufkirchen, DE |
| 3 | Salicylic acid | 100% | Sigma-Aldrich Chemie GmbH, |
| | | | Taufkirchen, DE |
| 4 | Silver nitrate | 100% | Sigma-Aldrich Chemie GmbH, |
| | | | Taufkirchen, DE |
| 5 | 1-Cetylpyridinium chloride | 100% | Sigma-Aldrich Chemie GmbH, |
| | | | Taufkirchen, DE |
| 6 | Hyamine 1622 | 100% | Sigma-Aldrich Chemie GmbH, |
| | | | Taufkirchen, DE |
| 7 | Preventol D 7 | 14% | Formulation of isothiazolones, |
| | | | Lanxess AG, Leverkusen, DE |
| 8 | Potassium iodide-iodine | 50% | Prepared by mixing equimolar |
| | complex | | amounts of potassium iodide and |
| | | | iodine in water, both chemicals |
| | | | from Sigma-Aldrich Chemie |
| | | | GmbH, Taufkirchen, DE |
| 9 | Propyl p-hydroxybenzoate | 100% | Sigma-Aldrich Chemie GmbH, |
| | | | Taufkirchen, DE |
| 10 | Hexamethoxymethylmelamine | 84% | Sigma-Aldrich Chemie GmbH, |
| | | | Taufkirchen, DE |
| 11 | Urotropin | 100% | Sigma-Aldrich Chemie GmbH, |
| | | | Taufkirchen, DE |
| 12 | 4-Hydroxybenzophenone | 100% | Sigma-Aldrich Chemie GmbH, |
| | | | Taufkirchen, DE |
| 13 | Curcumin | 100% | Sigma-Aldrich Chemie GmbH, |
| | | | Taufkirchen, DE |
| 14 | Preventol R 50 | 50% | Benzalkonium chloride, Lanxess |
| | | | AG, Leverkusen, DE |
| 15 | Preventol O extra | 100% | Sodium 2-phenylphenolate, |
| | | | Lanxess AG, Leverkusen, DE |
| 16 | Preventol SB extra | 100% | N-(4-Chlorophenyl)-N′-(3,4- |
| | | | dichloro-phenyl)urea, Lanxess |
| | | | AG, Leverkusen, DE |
| 17 | Preventol CMK pastilles | 100% | 4-Chloro-3-methylphenol, |
| | | | Lanxess AG, Leverkusen, DE |
| 18 | Glutaraldehyde | 50% | Sigma-Aldrich Chemie GmbH, |
| | | | Taufkirchen, DE |
| 19 | Formaldehyde | 37% | Sigma-Aldrich Chemie GmbH, |
| | | | Taufkirchen, DE |
| 20 | Camphor (racemic) | 100% | Sigma-Aldrich Chemie GmbH, |
| | | | Taufkirchen, DE |
| 21 | Menthol (racemic) | 100% | Sigma-Aldrich Chemie GmbH, |
| | | | Taufkirchen, DE |

### Example 1

### Preparation of a Nanourea Dispersion Without Active Compound

820.20 g of Bayhydur® VP LS 2336 and subsequently 0.32 g of Isofoam® 16 were added to a solution of 20.72 g of triethylamine in 4952 g of deionized water at 30° C. with vigorous stirring and stirred further. After 3, 6 and 9 hours, in each case a further 820.20 g of Bayhydur® VP LS 2336 and subsequently 0.32 g each of Isofoam 16 were added and the mixture was subsequently stirred at 30° C. for a further 4 hours. Afterwards, it was stirred under a vacuum of 200 mbar and at 30° C. for a further 3 hours and the resulting dispersion was bottled.

The white dispersion obtained had the following properties:

| | |
|---|---|
| Solids content: | 40% |
| Particle size (LKS): | 83 nm |
| Viscosity (viscometer, 23° C.): | <50 mPas |
| pH (23° C.): | 8.33 |

Charge determination: total charge 57±6 μeq/g, surface charge 15±1 μeq/g
Zeta potential (pH=8): 24.9±1.0

### Example 2

Subsequent Loading of a Nanourea with Active Compounds

For the preparation of active compound-containing nanourea dispersions, in each case 50 g of the nanourea dispersion from Example 1 were in each case treated with such a quantity of the active compounds 1 to 21 that an about 3% strength active compound concentration resulted based on the solids content. The mixture was vigorously stirred for 18 hours by means of a magnetic stirrer. After filtration of the resulting dispersion, 10 ml each of the sample obtained were filled into a dialysis tube and dialyzed twice against about one litre each of deionized water (altogether about 22 hours). The samples were withdrawn from the dialysis tube by means of a pipette and employed in the active compound test.

### Example 3

### Preparation of a Nanourea in the Presence of an Active Compound

410 g of Bayhydur® VP LS 2336 and 41.0 g of camphor were mixed in a reaction vessel with stirring. Subsequently, the mixture was dispersed at about 23° C. with vigorous stirring by addition of 1058 g of deionized water and treated with 0.04 g of Isofoam 16 and 2.59 g of triethylamine. Immediately thereafter, a vacuum of 200 mbar was applied and the mixture was stirred for about 10 hours; the temperature meanwhile rose in the course of this to about 30% C. The resulting dispersion was bottled.

The white dispersion obtained had the following properties:

| | |
|---|---|
| Solids content: | 27% |
| Particle size (LKS): | 93 nm |
| Viscosity (viscometer, 23° C.): | <50 mPas |
| pH (23° C.): | 6.98 |

### Example 4

### Preparation of a Nanourea in the Presence of an Active Compound

The procedure was as described in Example 3, but racemic menthol was employed instead of camphor.

The white dispersion obtained had the following properties:

| | |
|---|---|
| Solids content: | 28% |
| Particle size (LKS): | 86 nm |
| Viscosity (viscometer, 23° C.): | <50 mPas |
| pH (23° C.): | 7.90 |

### Example 5

### Test of the Active Compound-Loaded Nanourea Dispersions for Action Against Bacteria

Cells of Staphylococcus epidermidis 498 and Bacillus subtilis 168 are plated out on agar plates such that, after incubation overnight at 37° C., they have formed a visible cell lawn on the agar. The agar contained a complex nutrient-rich medium (Müller-Hinton medium, OD₆₀₀=0-1 set; 200 μl plated out per plate, dried at room temperature for one hour). A hole was in each case punched in the centre of the plates. Active compound-containing nanourea dispersions (100 μl), which had been prepared analogously to Example 2 and in which the active compound was present exclusively in bound form, were filled into this hole. After incubation overnight, the agar was examined for diffusing antibiotic active compounds in that the missing cell lawn around the punched-out hole can be seen (“inhibition halos”). These inhibition halos were compared with an agar plate without further additives and an agar plate with an active compound-free nanourea dispersion.

TABLE
Diameter of the inhibition halos in micrometres [minus the diameter
of the punched-out hole for active compound application]

| | | Inhibition halo in μm | |
|---|---|---|---|
| | | Staphylococcus | Bacillus |
| Experiment | Active compound | epidermidis 498 | subtilis 168 |
| A | without additives | 0 | 0 |
| B | Comparison: nanourea | 0 | 100 |
| | without active compound | | |
| | (from Example 1) | | |
| C | Silver nitrate | 400 | 300 |
| D | 1-Cetylpyridinium | 300 | 200 |
| | chloride | | |
| E | Preventol D 7 | 900 | 700 |

(C to E: dispersions of Example 2, B: dispersion of Example 1)

It was seen that, in comparison to the control experiments A and B, on use of active compound-modified nanourea dispersions (C to E) an antimicrobial action is present.

Furthermore, the active compound-loaded nanourea dispersions according to the invention have a controlled release profile based on the active compound contained therein in bound form. This can be seen by means of the antibacterial action detected, since, on account of the preceding dialysis, the dispersions employed themselves no longer contained any free unbound active compound and the antibacterial action can only occur by bound active compound being released again.

### Example 6

### Test of the Active Compound-Loaded Nanourea Dispersions for Action Against Bacteria

A test according to Example 5 was carried out in an overnight culture of Staphylococcus epidermidis (ATC 14990). The underlying active compound-loaded dispersions F to K were in turn prepared analogously to Example 2. As comparisons L to O, aqueous solutions having various concentrations of ciprofloxacin (Cipro) were additionally tested.

| | | Inhibition halo in mm |
|---|---|---|
| | | Staphylococcus epidermidis |
| Experiment | Active compound | (ATC 14990) |
| F | Acetylsalicylic acid | 13 |
| G | Salicylic acid | 38 |
| H | Potassium iodide-iodine | 14 |
| | complex | |
| I | Hexamethoxymethylmelamine | 13 |
| J | Preventol R 50 | 36 |
| K | Formaldehyde | 21 |
| L | Cipro 1 mM | 43 |
| M | Cipro 0.1 mM | 36 |
| N | Cipro 0.01 mM | 26 |
| O | Cipro 0.001 mM | 11 |

### Example 7

### Test for Determining a Delayed Release of Active Compound (Slow-Release Properties)

a) 900 g each of the nanourea dispersion from Example 1 were treated with 36 g of silver nitrate in a beaker with stirring. The dispersion was stirred at room temperature for 24 hours and subsequently stored in a closed bottle for 5 months.
1.04 g (corresponding to 0.04 g of silver nitrate) of the mixture were stirred into 40 g of Impranil DLN (80 minutes). Subsequently, a film is drawn on a glass plate using a doctor blade (gap: 210 μm) and dried at room temperature for one hour. The film was detached and a piece three×three cm in size was excised from the centre. The excised film was laid in 10 ml of deionized water in a screw-capped bottle such that the water completely flows around the film. After 24 hours, the water was changed in order to separate superficially attached silver ions.
Subsequently, the water was changed after 3, 7 and 51 days for new deionized water (counted from the first change of water) and in each case the concentration of silver ions was analysed.
b) The procedure was analogous to a), but the mixture of silver nitrate and nanourea dispersion was freshly prepared and directly employed further after the 24-hour mixing.
c) (Comparison experiment) The procedure was analogous to a), but instead of the mixture of silver nitrate and nanourea dispersion, 0.04 g of silver nitrate were directly mixed into the Impranil DLN dispersion.

TABLE
Extraction profile of the silver ions determined from
the concentration of silver ions [mg of Ag⁺ per
kg of the extraction solution, divided by the number of
days of the respective of the extraction period]

| | 3 days | 7 days | 51 days |
|---|---|---|---|
| a) | 0.1450 | 0.0525 | 0.0295 |
| b) | 0.1800 | 0.0525 | 0.0319 |
| c) (comparison) | 0.1400 | 0.1225 | 0.0113 |

With an identical amount of mixed silver ions, it was seen that, in the case of the comparison experiment c) without addition of nanoureas, the amount of released silver ions decreases distinctly more rapidly than in the case of the addition of the nanoureas. In the period between 7 and 51 days, in the comparison experiment c) in comparison to the experiments a) and b) only about one third of the silver ions is released. This shows that the antimicrobial action in the comparison experiment c) in comparison to the experiments a) and b) is considerably curtailed. In experiments a) and b), the desired delayed release of the silver nitrate ions is realized.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

1-15. (canceled)

16. A process for coating an article comprising
(1) applying a coating formulation comprising a biologically active compound-containing aqueous nanourea dispersion to an article, wherein said biologically active compound-containing aqueous nanourea dispersion comprises
A) nanourea particles formed by reacting a hydrophilized polyisocyanate with water in an aqueous medium, wherein said nanourea particles comprise urea structures of formula —NH—C(O)—NH—; and
B) at least one active compound added before, during, or after the formation of said nanourea particles in A); and
(2) drying and/or hardening said coating formulation to form a coating on said article;
wherein said nanoureas are not prepared from polyols.

17. The process of claim 16, wherein said aqueous medium in A) is a water-containing mixture containing at least 95% by weight of water.

18. The process of claim 16, wherein said hydrophilized polyisocyanate is a non-ionically hydrophilized polyisocyanate.

19. The process of claim 16, wherein said hydrophilized polyisocyanate is based on polyisocyanates having exclusively aliphatically or cycloaliphatically bonded isocyanate groups or mixtures thereof.

20. The process of claim 16, wherein A) is performed in the presence of one or more catalysts.

21. The process of claim 20, wherein said one or more catalysts is a tertiary amine.

22. The process of claim 16, wherein the molar ratio of NCO groups of said hydrophilized polyisocyanates to water is from 1:30 to 1:10.

23. The process of claim 16, wherein said hydrophilized polyisocyanates are incorporated into said aqueous medium in portions.

24. The process of claim 16, wherein said nanourea particles comprises one or more biologically active compounds in an amount of from 5 to 50% by weight based on the total solids content of said nanourea particle.

25. A coating obtained by the process of claim 16.

26. An article coated with the coating of claim 25.

27. The article of claim 26, wherein said article is selected from the group consisting of catheters, tubes, vessels, orifices, implants, artificial organs, prostheses, vascular prostheses, visual aids, endoscopes, and wound coverings.
